(19) United States
(12) Patent Application Publication
(10) Pub. No.: US 2022/0040148 A1
(43) Pub. Date: Feb. 10, 2022

(54) COMBINATION DRUG FORMULATIONS INCLUDING ROTIGOTINE AND AN ACETYLCHOLINESTERASE INHIBITOR FOR THE TREATMENT OF NEURODEGENERATIVE DISEASES

(71) Applicant: Alzheimer's Drug Discovery Foundation, New York, NY (US)

(72) Inventors: Howard Fillit, New York, NY (US); Giacomo Koch, Rome (IT); Alessandro Martorana, Rome (IT); Alessio Travaglia, San Diego, CA (US)

(21) Appl. No.: 17/375,949

(22) Filed: Jul. 14, 2021

Related U.S. Application Data
(60) Provisional application No. 63/051,556, filed on Jul. 14, 2020.

(51) Int. Cl. A61K 31/381; A61K 9/00; A61K 31/13; A61P 25/28

(52) U.S. Cl. CPC A61K 31/381; A61P 25/28; A61K 31/13; A61K 9/0014

(57) ABSTRACT

This disclosure relates to the pharmacological treatment of apathy and loss of executive function in treatment of neurodegenerative diseases by administration of a combination drug formulation including rotigotine and an acetylcholinesterase inhibitor.

### CROSS REFERENCE TO RELATED APPLICATIONS

This application claims the benefit of U.S. Provisional Application No. 63/051,556 filed on Jul. 14, 2020, the entire disclosures of which is incorporated herein by reference for all purposes.

### TECHNICAL FIELD

The present disclosure relates to the treatment of neurodegenerative disorders. More specifically, the present disclosure relates to the pharmacological treatment of apathy and loss of executive function in such disorders. Further, the present disclosure relates to the pharmacological treatment of neurodegenerative diseases by administration of a combination drug formulation including rotigotine and an acetylcholinesterase inhibitor.

### BACKGROUND

In recent decades, evidence strengthened the concept that the impairment of dopaminergic transmission may contribute to cognitive dysfunction in Alzheimer's disease. Dopamine is a key neuromodulator affecting several distinct steps of synaptic transmission, playing an important role in the control of high cognitive functions such as memory, learning and decision-making. Post-mortem studies revealed marked loss of dopamine receptors in the temporal and frontal lobes of Alzheimer's disease brains, showing a relationship between decreased levels of D2-like receptor and Alzheimer's disease pathophysiology.

These neuropathological findings were confirmed by in vivo investigations with positron emission tomography. The use of dopaminergic drugs such L-dopa or selegiline in patients with different stages of Alzheimer's disease resulted in some controversial results. More recently, experimental studies in animal models of Alzheimer's disease showed that dopaminergic agonists may reduce amyloid deposition and improve memory, and that the degeneration of dopaminergic neurons in the ventral tegmental area contributes to memory deficits. Recent studies have suggested that in early stages of Alzheimer's disease, patient's dopaminergic agonists improve cholinergic transmission and cortical plasticity, likely by acting on the dopaminergic projections over the frontal cortex. Based on these studies, therapies based on dopaminergic stimulation in patients with mild to moderate Alzheimer's disease may have a relevant clinical impact on cognitive impairment in Alzheimer's disease patients.

It would be advantageous to provide a method of treating apathy and loss of executive function brought about by neurodegenerative disorders using a dopaminergic-agonist, such as rotigotine, as adjunctive therapy to standard treatment with acetylcholinesterase inhibitors in patients with mild to moderate Alzheimer's disease.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

In one aspect, the present disclosure relates to a method for relieving symptoms of dementia associated with neurodegenerative diseases including the step of administering a transdermal combination drug formulation comprising a first therapeutically effective dose of a dopaminergic agonist such as rotigotine and a second complementary dose of an acetylcholinesterase inhibitor. In embodiments, the neurodegenerative disease comprises Alzheimer's disease, frontotemporal dementia or Lewy body disease, vascular dementia, or a combination of such findings. In embodiments, the acetylcholinesterase inhibitor comprises donepezil, galantamine huperzine or rivastigmine.

In another aspect, the present disclosure relates to a method for relieving agitation in a patient with a neurodegerative disease including the step of administering to the patient a transdermal combination drug formulation that delivers: (a) rotigotine in a dose from 2 mg/24 hours to 8 mg/24 hours; and (b) an acetylcholinesterase inhibitor in a dose from 5 mg to 23 mg per 24 hours. In embodiments, the neurodegenerative disease comprises Alzheimer's disease, frontotemporal dementia or Lewy body disease, vascular dementia, or a combination of such findings. In embodiments, the acetylcholinesterase inhibitor comprises donepezil, galantamine huperzine or rivastigmine.

### BRIEF DESCRIPTION OF THE DRAWINGS

A more complete understanding of the presently disclosed concepts and illustrative embodiments may be acquired by referring to the following description, taken in conjunction with the figures of the accompanying drawings wherein:

FIG. 1 is a flow chart depicting randomization, trial group assignment, and follow-up of participants in a scientific study of a combination drug formulation in accordance with illustrative embodiments of the present disclosure;

FIG. 2 depicts the Estimated Mean Change from Baseline in the ADAS-Cog (Panel A), FAB (Panel B), ADCS-ADL (Panel C), and NPI (Panel D) scores over 24 Weeks in a scientific study of a combination drug formulation in accordance with illustrative embodiments of the present disclosure;

FIG. 3 depicts changes in global mean field power (GMFP) (upper panels A-B) and oscillatory activity (middle and lower panels C-F) evoked from the left dorsolateral prefrontal cortex in the rotigotine and placebo groups before and after completion of the scientific study of a combination drug formulation in accordance with illustrative embodiments of the present disclosure;

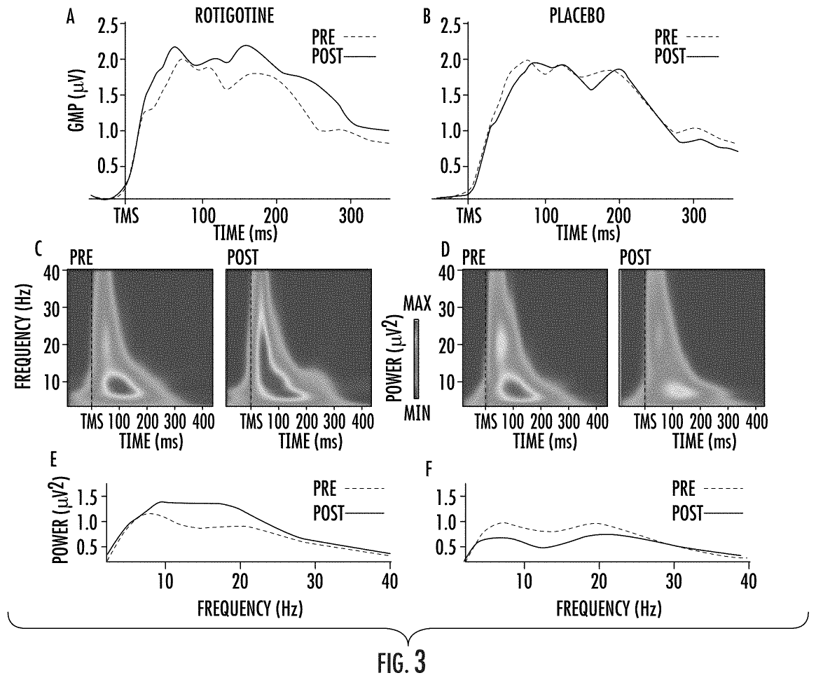

FIG. 4A depicts oscillatory in a group of Alzheimer's Disease patients after 24 weeks of treatment with Rotigotine in combination with rivastigmine;

FIG. 4B depicts oscillatory activity in a group of Alzheimer's Disease patients after 24 weeks of treatment with rivastigmine in combination with a placebo; and

FIG. 5 is a graph depicting the differences in oscillatory activity data between Alzheimer's Disease patients treated with a combination of rotigotine and rivastigmine, and Alzheimer's Disease patients treated with a placebo.

### DETAILED DESCRIPTION

A method of treating apathy and loss of executive function brought about by neurodegenerative disorders using a dopaminergic-agonist, such as rotigotine, as adjunctive therapy to standard treatment with acetylcholinesterase inhibitors in patients with mild to moderate Alzheimer's disease is described herein.

Standard treatment of neurodegenerative disorders brought on by Alzheimer's disease often involves the use of drugs including acetylcholinesterase inhibitors. These drugs do not target the beta-amyloid plaques and neurofibrillary tangles associated with Alzheimer's Disease (AD), but function by inhibiting the acetylcholinesterase enzyme and thus reducing the rate at which acetylcholine is broken down in the neural synaptic cleft. This net increase in free acetylcholine is associated with increased memory and cognitive function.

Acetylcholinesterase and cholinesterase inhibitors suitable for combination drug formulations in accordance with this disclosure may include, but are not limited to, physostigmine, tacrine and tacrine analogues, fasiculin, metrifonate, heptylphysostigmine, norpyridostigmine, norneostigmine, huperazine, rivastigmine, galantamine, donepezil and pro-drugs of any of these in which the inhibitor is modified in accordance with principles of pro-drug construction known in the art. Examples of such modifications include the introduction of hydrophilic or lipophilic groups to enhance solubility, or penetration through cell membranes, respectively, in some embodiments; cholinesterase inhibitors are acetylcholinesterase inhibitors, particularly those which are capable of crossing the blood brain barrier.

Additional acetylcholinesterase inhibitors that may be suitable for combination drug formulations in accordance with this disclosure are described for example in U.S. Patent Application No. US 2018015109A1, the disclosure of which is incorporated herein by reference in its entirety.

In alternative embodiments, other suitable drugs for treating Alzheimer's Disease, and other similar neurodegenerative diseases may be used in combination with Rotigotine to create combination drug formulations in accordance with this disclosure.

For example, in embodiments Rotigotine may be combined with the Alzheimer's Disease drug Memantine (I-amino-3,5-dimethyl adamantane), which is disclosed, e.g., in U.S. Pat. Nos. 4,122,193; 4,273,774; and 5,061,703, a systemically-active uncompetitive NMDA receptor antagonist having low to moderate affinity for the receptor and strong voltage dependency and rapid blocking/unblocking kinetics. Memantine hydrochloride is approved for the treatment of moderate to severe dementia of the Alzheimer's type in the United States and is available as Namenda® (5 and 10 mg BID immediate release tablets) and Namenda XR® (28 mg once-daily extended release capsules).

Combination drug formulations in accordance with this disclosure can be administered via any of the accepted modes of administration or agents known in the art. The drugs may be administered, for example, orally, nasally, parenterally (intravenous, intramuscular, or subcutaneous), topically, or transdermally. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, for example, in unit dosage forms suitable for simple administration of precise dosages. A particular route of administration is oral, particularly one in which a convenient daily dosage regimen can be adjusted as desired.

In particular embodiments, combination drug formulations in accordance with this disclosure combine one or more acetylcholinesterase inhibitors with Rotigitine.

Rotigotine is the international non-proprietary name (EN) of the compound (−)-5,6,7,8-tetrahydro-6-[propyl-[2-(2-thienyl)ethyl]amino]-1-naphthalenol having the structure shown below.

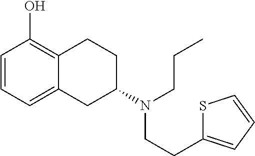 [SMILES: CCCN(CCc1cccs1)[C@H]1CCc2c(O)cccc2C1]

Rotigotine is a non-ergolinic D1/D2/D3 dopamine agonist that resembles dopamine structurally and has a similar receptor profile but a higher receptor affinity.

In contrast to other non-ergolinic dopamine agonists, rotigotine has significant D1 activity, which may contribute to a greater physiological action.

In contrast to ergolinic compounds, rotigotine has a very low affinity for 5-HT_{2B} receptors and thus a low risk of inducing fibrosis.

Actions on non-dopaminergic receptors (such as 5-HT_{1A} agonism and A2B antagonism) may contribute to other beneficial effects, such as antidyskinetic activity, neuroprotective activity and antidepressive effects.

Currently, the most commonly used pharmaceutical product containing rotigotine is a transdermal therapeutic system (TTS). Neupro® (sold by UCB Pharma GmbH) is formulated as a once-daily TTS and provides a constant delivery to the skin of between 1 to 8 mg/24 hours of rotigotine. Suitable pharmaceutical products for use in combination drug formulations in accordance with this disclosure are described in, for example, U.S. Pat. Nos. 6,669,498, 6,884,434, 7,413,747, 8,246,979, 8,246,980, and 8,617,591, the entire disclosures of which are incorporated herein by reference.

A scientific study was conducted to evaluate methods of treating apathy and loss of executive function brought about by neurodegenerative disorders using a dopaminergic-agonist, such as rotigotine, as adjunctive therapy to standard treatment with acetylcholinesterase inhibitors in patients with mild to moderate Alzheimer's disease. The details of the scientific study are described below in connection with embodiments in accordance with the present disclosure.

### Methods

A study was designed in which patients were eligible if they had an established diagnosis of probable Alzheimer's disease according to National Institute of Neurological and Communicative Disorders and Stroke and the Alzheimer's Disease and Related Disorders Association criteria; aged greater than 50 and less than 85 years; had a Clinical Dementia Rating score of 0.5-1 and Mini Mental State Examination (MMSE) score of 18-26 at screening indicating mild to moderate Alzheimer's Disease; had one caregiver; had been treated with acetylcholinesterase inhibitor for at least 6 months; had performed lumbar puncture for cerebrospinal fluid biomarkers analysis for diagnostic purposes. Patients underwent medical and neurologic evaluations, including magnetic resonance imaging or computed tomography. Patients were excluded if they had extrapyramidal signs, history of stroke, other neurodegenerative disorder, psychotic disorders and if they had been treated six months before enrollment with antipsychotics, antiparkinsonian, anticholinergics and antiepileptic drugs. The trial was approved by the review board and ethics committee at the Santa Lucia Foundation and was conducted in accordance with the principles of the Declaration of Helsinki and the International Conference on Harmonisation Good Clinical Practice guidelines. All patients or their parents or legal representatives provided written informed consent. Patients could withdraw at any point without prejudice. This report followed the CONSORT reporting guideline for randomized studies. The trial was registered with ClinicalTrials.gov and number NCT03250741.

### Randomization and Masking

The study was a monocentric, randomized, double-blind trial of rotigotine versus placebo in mild to moderate Alzheimer's disease patients as add-on to treatment with acetylcholinesterase inhibitors. The trial comprised a 24-week treatment period with a 1 week of dose escalation of transdermal patches of rotigotine at 2 mg per day and 23 weeks of dose maintenance of transdermal patches of rotigotine at 4 mg per day. The dose of rotigotine used in the trial was recommended by an independent data and safety monitoring committee whose members reviewed data from safety evaluation and identified a safe maximum dose not associated with unacceptable side effects. A 4 mg dosage was chosen to be effective in modulating cholinergic activity and cortical plasticity in Alzheimer's disease patients, while ensuring no relevant side effects were experienced.

### Trial Procedures

After recruitment and baseline assessments, patients were randomly assigned in a 1:1 ratio to receive rotigotine or matching placebo in addition to their stable drug regimen with acetylcholinesterase inhibitors therapy. All treatments were administered for 24 weeks with no interruptions. Rotigotine was administered through a 4 mg transdermal patch (commercially available as Neupro®, sold by UCB pharma). After having started with a 2 mg patch for 1 week, transdermal patches of rotigotine had a release surface area of 10 or 20 cm² and contained 4.5 or 9 mg of rotigotine to release respectively 2 or 4 mg during a 24-hour period when applied to intact skin. The placebo transdermal patch contained in cardboard packaging was identical to the rotigotine except for the absence of rotigotine. The efficacy assessments were rated at baseline for enrolled subjects and caregivers and repeated at weeks 24 (or upon early termination) by assessors/raters who were blinded in respect to the assignment group.

### Outcomes Measures

The primary end-point was the change at 24 weeks from baseline on the Alzheimer's Disease Assessment Scale-Cognitive Subscale (ADAS-Cog). The ADAS-Cog measures severity of impairment in various cognitive domains (memory. language. orientation, praxis, and executive functioning). The scale has a score range of 0 to 70 points, with higher scores indicating worse performance. The scale is analyzed as a continuous measure. The intention-to-treat analysis set included all patients who had post-baseline efficacy data. The secondary key end-point measures were the change at 24 weeks from baseline on the Activities of Daily Living (ADCS-ADL), the Frontal Assessment Battery (FAB) and the Neuropsychiatric Inventory (NPI). Transcranial magnetic stimulation in combination with electroencephalography (TMS-EEG) was used to monitor the effects of treatment on frontal lobe cortical activity. This TMS-EEG approach was selected because it allowed for assessment of the neurophysiological state of a specific cortical area as an elective method for the assessment of neural processing through objective measurements of cortical activity, in terms of both cortical excitability and oscillatory dynamics. Hence, as biomarkers measured neurophysiological changes induced by dopamine-agonist over the left dorsolateral prefrontal cortex (DLPFC) and the left posterior parietal cortex (PPC) by evaluating the cortical excitability and oscillatory activity evoked by single-pulse TMS combined with EEG recordings.

For each patient, eighty single TMS pulses were applied over each stimulation site (left DLPFC and left PPC), during an EEG recording with open eyes, with an intensity of 90% of the resting motor threshold. A TMS-compatible EEG equipment (BrainAmp 32MRpluls, commercially available from BrainProducts GmbH, Munich, Germany) was used to record the EEG activity from 29 scalp sites positioned according to the 10-20 International System. TMS-compatible Ag/AgCl pellet electrodes were mounted on an elastic cap, while additional electrodes were used as ground and reference. Eye movements were detected by recording the electrooculogram (EOG). The EEG and EOG signals were band-pass filtered at 0.1-1000 Hz and digitized at a sampling rate of 5 kHz. Skin/electrode impedance was maintained below 5 kΩ.

TMS-EEG data was analyzed off-line (Brain Vision Analyzer, Brain Products GmbH, Munich, Germany), with different approaches both in spatio/temporal-domain for evaluating cortical excitability changes and in time/frequency domain for evaluating cortical oscillatory changes.

Two sets of outcome measures were obtained assessing cortical excitability (global mean field power, GMFP) and cortical oscillatory activity. At each clinic visit (or upon early termination), adverse events (AEs) were recorded, vital signs measured and physical and neurological examination performed. An independent Data Monitoring Committee monitored the patients' safety according to the Data Monitoring Committee Charter.

### Statistical Analysis

A total of 94 randomly assigned patients (47 per group) were planned on the basis of a previous study in which effects of rotigotine on cortical plasticity and cognitive functions in a small sample of Alzheimer's disease patients were assessed. (See Koch G, Di Lorenzo F, Bonni S, et al. Dopaminergic modulation of cortical plasticity in Alzheimer's disease patients. Neuropsychopharmacology 2014; 39:2654-2661). In that pilot study, ADAS-Cog data was not collected, however a significant difference was observed in pre-post (12 weeks) treatment with rotigotine in patients in both MMSE and F AB. Adopting a power computation based on a two-tailed paired t-test, with type I error alpha=0.05 and a plausible correlation between pre-post measured variables of 0.7, the FAB effect size observed in the pilot study equal to 0.42 (obtained as post-pre FAB means over pooled standard deviation, see study protocol in Supplementary materials for details) requires a minimum sample of n=46 for reaching a power of 0.8. For MMSE (for which the effect size was 0.48), this sample size allows to reach a power of 0.9. The minimum total sample size was then augmented up to N=92 considering the matched placebo group. Randomization was performed and assigned by a statistician working in an independent institution. In order to obtain homogeneous and balanced study groups in terms of age, sex and APOE carriers, an adaptive randomization was adopted. Normality assumption of end-points variables were assessed by inspection of the distribution plots and by KolmogoTov-Smirnov and Shapiro-Wilk tests. The longitudinal assessment of the end-points across groups were performed through generalized linear mixed model (GLMM) for repeated measures with random intercept and random slope to account for individual differences at baseline as well as for individual changes during the follow-up. GLMM were applied to ADAS-Cog-11 and to the other efficacy outcome measures, ADCS-ADL, FAB and NPI, as dependent variables and “group”, “time” and “groupxtime” interaction as independent factors. In detail, GLMM for Gaussian data with identity link function were applied for ADAS-Cog-11, ADCS-ADL and FAB, whereas GLMM for Poisson data, with log-link function, was used for NPI. The GLMM on MMSE, ADAS-Cog-11 and FAB were adjusted for age and education. To evaluate the treatment effects of TMS-EEG data repeated-measures ANOVAs with between-subjects factor “group” and within-subject factors “time” were used. All statistical analyses were performed with IBM SPSS Statistics for Windows, version 25 (IBM Corp., Armonk, N.Y., USA). Statistical tests were 2-tailed, and P<0.05 was considered statistically significant.

### Results

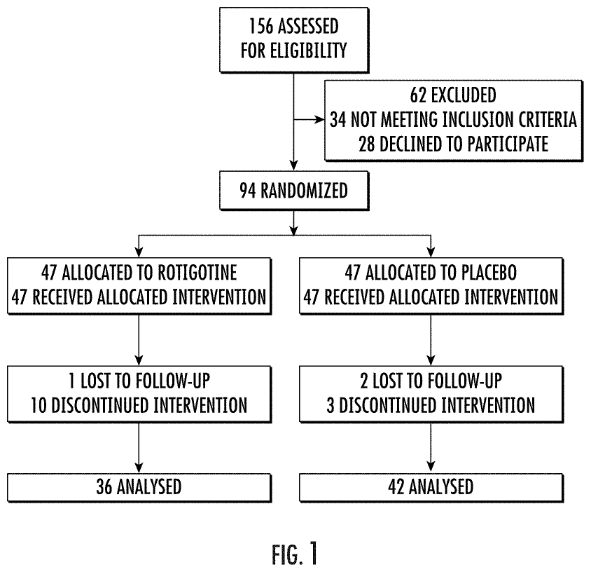

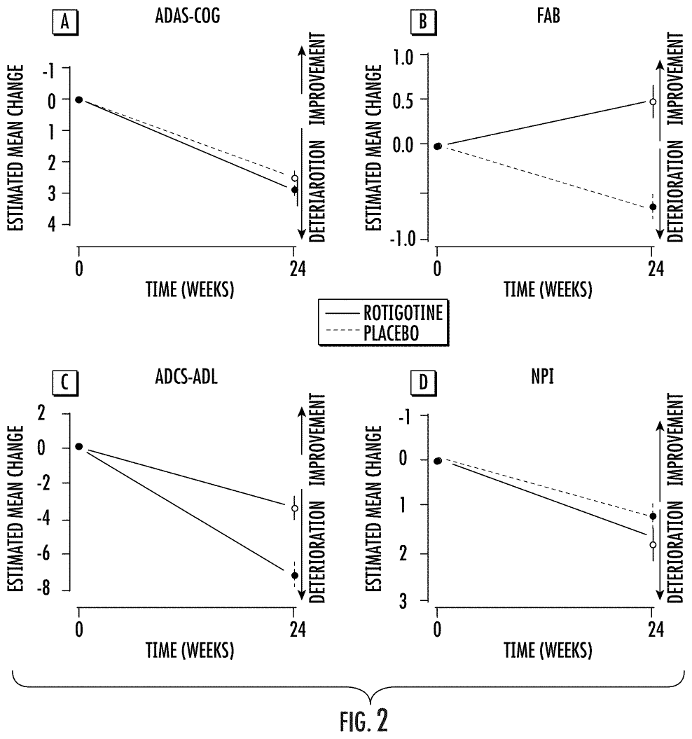

156 patients were screened and 94 underwent randomization (FIG. 1). The mean age of the total sample of patients was 73.9 years (SD=5.6, range 55 to 83), and 61% were female. Patients had a mean MMSE raw score at baseline of 23.2 (SD=2.4). The percentage of patients screening positive as carriers for at least one APOE €4 allele was 62%. The baseline patients' demographics and clinical characteristics did not differ between the rotigotine and placebo groups in terms of age, education, time since diagnosis of Alzheimer disease, time since current cholinesterase inhibitor treatment initiated, APOE €4 carrier, MMSE, ADAS-CogII, F AB, ADCS-ADL and NPI scores (Table 1). A total of 16 patients withdrew from the trial before completion (11 in the rotigotine group and 5 in the placebo group). A total of 78 patients (83%) completed the treatment period (FIG. 1). Based on previous pilot study, 78 patients are enough to reach a power of 0.8 considering an effect size equal to 0.48 for both MMSE and FAB measures. The mean baseline ADAS-Cog-11 total score was 19.8 (S0=6.4) for the rotigotine group, and 18.7 (S0=6.5) for the placebo group. There were no significant differences (baseline vs. week 24) in the cognitive performance as measured by the ADAS-Cog-11 total score in the rotigotine groups compared with placebo (Table 2). GLMM for repeated measures on ADAS-Cog-11 scores (adjusted for age and education) did not show any significant result in terms of group effect (p=0.54), time (p=0.71) and timex group (p=0.82) interaction, although estimated values showed a general worsening of cognitive performance of patients over time. The GLMM estimated mean change in ADAS-Cog-11 score was 2.92 for rotigotine (95% confidence interval (CI) [2.51, 3.33 J) and 2.66 for placebo group (95% CI [2.31, 3.01]) (FIG. 2A).

TABLE 1
Baseline Patients Demographics and
Clinical Characteristics at baseline.

| | rotigotine | placebo |
|---|---|---|
| | group | group |
| | N = 47 | N = 47 |
| Age, mean (SD) | 73.4 (5.8) | 74.3 (5.5) |
| Women, Female, N (%) | 31 (66%) | 27 (57%) |
| Education, years, mean (SD) | 8.5 (4.2) | 9.4 (4.3) |
| Time since diagnosis of | 1.3 (0.3-1.9) | 1.1 (0.4-1.8) |
| Alzheimer disease, median | | |
| (IQR), y | | |
| Time since current | 0.9 (0.6-1.2) | 0.8 (0.3-1.1) |
| cholinesterase inhibitor | | |
| treatment initiated, median | | |
| (IQR), y | | |
| APOE e4 carrier No (%) | 28 (60%) | 30 (64%) |
| MMSE raw score, mean (SD) | 22.9 (2.3) | 23.6 (2.4) |
| ADAS-Cog raw score, mean | 19.8 (6.4) | 18.7 (6.5) |
| (SD) | | |
| FAB raw score, mean (SD) | 11.4 (3.0) | 12.1 (3.0) |
| ADCS-ADL score, mean (SD) | 61.0 (12.6) | 62.8 (10.4) |
| NPI score, mean (SD) | 12.4 (9.9) | 12.8 (11.6) |
| UPDRS III score, mean (SD) | 2.6 (1.8) | 2.8 (1.6) |

TABLE 2
Change in Primary and Secondary Outeomes from Baseline to
Week 24: GLMM estimated effects

| | Estimated change from | | | | | | | |
|---|---|---|---|---|---|---|---|---|
| | baseline | | | | | | Group x | |
| | Rotigotine | Placebo | Group | | | | Time | |
| | Mean | Mean | Effect | | Time effect | | effect | |
| Outcomes | [95% CI] | [95% CI] | F value | p value | F value | p value | F value | p value |
| Primary | | | | | | | | |
| outcome | | | | | | | | |
| ADAS-Cog | 2.92 [2.51, | 2.66 [2.31, | F_{1,163} = 0.37 | p = 0.545 | F_{1,163} = 0.14 | p = 0.713 | F_{1,163} = 0.05 | p = 0.822 |
| score# | 3.33] | 3.01] | | | | | | |
| Secondary | | | | | | | | |
| outcomes | | | | | | | | |
| ADCS- | −3.32 [−4.02, | −7.24 [−7.84, | F_{1,164} = 0.02 | p = 0.881 | F_{1,164} = 0.17 | p = 0.680 | F_{1,164} = 4.24 | p = 0.041* |
| ADL score | −2.62] | −6.64] | | | | | | |
| FAB score# | 0.48 [0.31, | −0.66 [−0.80 | F_{1,164} = 0.04 | p = 0.843 | F_{1,164} = 0.001 | p = 0.976 | F_{1,164} = 5.99 | p = 0.015* |
| | 0.65] | −0.52] | | | | | | |
| NPI total | 1.64 [1.06, | 1.26 [0.77, | F_{1,164} = 0.01 | p = 0.927 | F_{1,164} = 0.02 | p = 0.890 | F_{1,164} = 0.05 | p = 0.818* |
| score# | 2.22] | 1.75] | | | | | | |

The analysis of secondary outcomes showed that there were significant differences between the rotigotine group and the placebo group for the F AB and ADCS-ADL but not for the NPI scores (Table 2). The GLMM estimated mean change in F AB score was 0.48 for rotigotine (95% CI [0.31, 0.65 and −0.66 for placebo group (95% CI [−0.80, −0.52]), showing that frontal lobe functions improved in rotigotine group as compared to placebo (interaction effect p=0.01) (FIG. 2B). The baseline mean of AOCS-ADL total score was 61.0 (SD=12.6) for the rotigotine group and 62.8 (SD=10.1) for the placebo group. Estimated mean change in ADCS-ADL scores was −3.32 for rotigotine (95% CI [−4.02, 2.62]) and −7.24 for placebo group (95% CI [−7.84, −6.64), showing an advantage of the rotigotine with respect to placebo (interaction effect: p=0.04) (FIG. 2C). The baseline mean for NPI total score was 12.4 (SD=9.9) for the rotigotine group, and 12.8 (SD=I 1.6) for the placebo group. Estimated mean change in NPI score was 1.64 for rotigotine (95% CI [1.06, 2.22) and 1.26 for placebo group (95% Cl [0.77, 1.75]) revealing no significant effects between the groups during follow up (FIG. 2D).

After 24 weeks of treatment, a significant increase of DLPFC activity was measured by GMFP in the rotigotine group (n=20) as compared to placebo group (N=20) (p=0.002). There was also a notable increase of DLPFC oscillatory activity in the rotigotine group as compared to placebo group (p=0.01). This effect was site specific, since no change in cortical activity was observed when TMS pulses were applied over the PPC (see supplementary Figure S2).

Adverse events were more common with rotigotine than with placebo. In total sixteen patients dropped out of which 11 patients assigned to rotigotine treatment and 5 patients to placebo (p=0.17). In the rotigotine group two patients reported allergy to the patch, one had visual hallucinations, one had pneumonia, three nausea and dizziness, one sleep disorders, one anxiety, one implanted with PMK and one declined to continue. In the placebo group one had pneumonia, one cervical pain, one had diagnosis of kidney tumor, one arrhythmia and one refused to continue.

### Discussion

The results of a dopamine-agonist treatment with rotigotine in patients with mild-to-moderate Alzheimer's disease is discussed below. In this trial, a daily dose of rotigotine shows no benefit with respect to the primary clinical outcome as measured by change in the ADAS-Cog-11 score from baseline to week 24, as compared with placebo. Indeed, rotigotine at a relatively low dosage is safe and well tolerated in patients with mild to moderate Alzheimer's disease. Adverse events were more common with rotigotine than with placebo but were similar to those seen in randomized-controlled trials testing rotigotine in patients with mild Parkinson's disease of comparable duration. Moreover, rotigotine did not induce any relevant behavioral side effect as revealed by NPI scores analysis. Notably, Alzheimer's disease patients enrolled for the current study were in the early phase of the disease and did not show any extrapyramidal sign such as tremor or rigidity. In agreement with previous studies showing that extrapyramidal symptoms are more likely to appear in the later stages of AD, patients enrolled in the present study did not show a significant rate of mild parkinsonism at the earlier stages of AD, as confirmed by the UPDRS scale assessment (Table 1).

The primary outcome analysis showed that rotigotine administration had no effects on memory and other cognitive tasks, as measured by ADAS-Cog-11. However, secondary outcome analysis showed a clear and remarkable effect on cognitive functions highly related to the frontal lobe. The effects of rotigotine on frontal lobe functions was evaluated, as dopamine largely modulates frontal cortex activity, and it has been shown that treatment with rotigotine induces an improvement of cortical plasticity in the frontal cortex in patients with mild Alzheimer's disease.

In the current trial, rotigotine improved cognitive functions highly related to the frontal lobe in Alzheimer's disease patients during 24 weeks, while these cognitive functions declined in patients treated with placebo. Moreover, rotigotine was efficacious in reducing the decline of functional impairment. The study showed an effect on the autonomies of daily living as compared to group of patients assuming placebo, suggesting that use of rotigotine could have a potential role in treating functional impairment since the early stages of the disease.

Besides memory impairment, decline in cognitive functions related to the frontal lobe activity and in everyday living activities represent the key features of the Alzheimer's disease progression. Executive functions play a crucial role for coping with the changing demands of everyday life, and have been related to the frontal lobes activity. The preservation of everyday living activities is closely related to executive functions and their impairment leads to early loss of independence, shifting many daily responsibilities to caregivers and increasing their burden. In this regard, everyday living activities impairment in Alzheimer's disease patients has been associated with global pathologic changes and frontal hypometabolism. Therefore, treating the impairment of frontal cognitive impairment represent one of the main targets for future pharmacological interventions. Apart from the positive effects on cognitive functions highly related to the frontal lobe, rotigotine also induced a remarkable increase of prefrontal cortex activity, as indexed by TMS-EEG recordings. Additionally, treatment with rotigotine also enhanced the evoked EEG response to TMS leading to an increase in oscillatory activity in the range of alfa and beta frequency.

Prolonged exposure to AR progressively impairs the physiological release of dopamine in the prefrontal cortex and hippocampus, contributing to the impairment of attention, memory, and executive functions. Magnetic resonance imaging showed that volume and connectivity of the ventral tegmental area are linked to cognitive impairment in patients with mild Alzheimer's disease. Notably, the ventral tegmental area is the major source of dopaminergic projections directed towards the prefrontal cortex through meso-cortical fibers. The combined clinical and TMS-EEG findings indicate that increasing dopaminergic neurotransmission with rotigotine likely enhances frontal lobe activity by acting on meso-cortical dopaminergic projections.

Despite the improvement of cognitive functions highly related to the frontal lobe, no effect on memory was observed, as also revealed by the analysis of ADAS-Cog-11 sub-items. It is possible to suppose that the association between dopamine agonists and cholinesterase inhibitors could have masked measurable effects on memory tasks. On the other hand, the medial temporal lobe is a site of complex pathological mechanisms linking neurodegeneration with neuroinflammation that likely begin long before the cognitive decline appears, making negligible the contribution of dopaminergic neurotransmission in patients with moderate Alzheimer's disease. Moreover, due to the relative low number of patients enrolled, the present study did not take in account the potential influence of APOE genotype and cognitive reserve.

While no cure or disease-modifying treatment is currently available for Alzheimer's disease and recent attempts with novel disease-modifying drugs have been ineffective. The most frequently prescribed treatment for Alzheimer's disease are acetylcholinesterase inhibitors.

The presently described compositions and methods involving the use of dopaminergic agonists such as rotigotine in combination with acetylcholinesterase inhibitors is safe in patients with mild to moderate Alzheimer's disease. Treatment with rotigotine reduces symptoms related to frontal lobe cognitive dysfunction and delay the impairment of autonomies of daily living.

It is well known that multiple factors contribute to the pathogenesis of AD, including amyloid-6 deposition, tau accumulation, microglia- and astrocyte-mediated inflammation, loss of neurons and synapses, and altered network oscillations. AD patients show a remarkable reduced power of oscillations in the gamma frequency band, a phenotype that may be replicated in AD mouse models (See for example, Gillespie et al., 2016; laccarino et al., 2016; Verret et al., 2012). Such altered oscillations have been related with cognitive dysfunction.

In an embodiment of the present disclosure, rotigotine is used in combination with rivastigmine. The details of the methods used to test this drug combination are described below in connection with embodiments in accordance with the present disclosure.

A study was performed to investigate the effects of rotigotine in combination with rivastigmine on the power of oscillations measured in the prefrontal cortex of AD patients. In the study, AD patients were treated with a therapeutically effective dose in accordance with the present disclosure of a drug combination of rotigotine and rivastigmine for a period of 24 weeks. At the end of the treatment period, changes in oscillatory activity were evaluated by using neurophysiological methods based on the co-registration of transcranial magnetic stimulation (TMS) with electroencephalography (EEG). Cortical oscillations were evoked by pulses of TMS applied over the left dorsolateral prefrontal cortex (DLPFC) while recording EEG.

To assess the effect of rotigotine over the oscillatory activity of the left DLPFC, a time/frequency decomposition was performed for each epoch based on Morlet wavelet. To track the significant spectral modulation induced by TMS, TRSP was extracted at the signal trial level using a number of cycles that linearly increase from 2 at the lowest frequency, to 20 at the highest. TRSP were expressed in decibel relative to the mean power in the baseline interval from −800 to −200 ms. Significant ERSP's were detected using a bootstrap approach, which has the advantage of avoiding a priori assumptions about data while correcting for multiple comparisons. One-tailed t-tests were computed over the 46 frequencies (ranging from 4 to 50 Hz) and the 200 time points (ranging from −1000 to 1000 ms). Statistical significance was set at p=0.05 (2000 permutations).

A measure of how TMS modulates the power spectral activity of the stimulated cortical regions after 24 treatment with RTG in combination with rivastigmine was then taken.

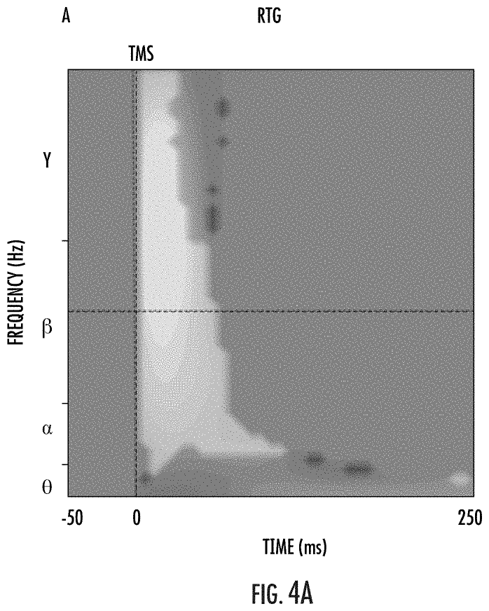

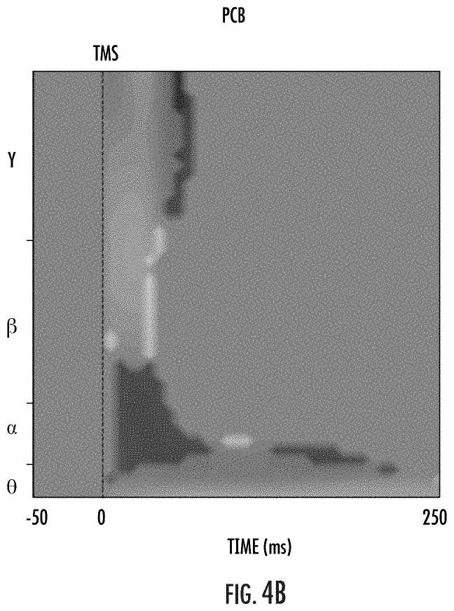

The data indicated that rotigotine in combination with rivastigmine significantly enhanced oscillations in the DLPFC in the range of high frequencies (from 22 to 32 Hz) (all ps<0.05) (FIG. 1, panel A), as compared to the group of patients treated with rivastigmine in combination with placebo condition (PCB) (See FIG. 4A)

This data shows that the combination of rotigotine and rivastigmine results in an increased oscillatory activity that is generally specific for the higher frequency bands (See FIG. 4A). Such an effect is seen only when rotigotine is combined with rivastigmine, while no change in oscillations was observed when rivastigmine was combined with PCB (See FIG. 4B)

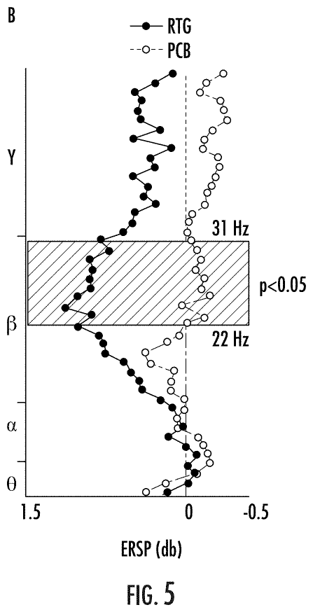

For clarity, FIGS. 4A-B depict changes in local cortical oscillatory activity induced by rotigotine in combination with rivastigmine as compared to rivastigmine in combination with placebo (PCB) measured in the left dorsolateral prefrontal cortex (DLPFC) using combined transcranial magnetic stimulation (TMS) and electroencephalography (EEG) co-registration. FIG. 4A displays the increase of oscillations more evident in the high frequency band in the group of AD patients after 24 weeks of treatment with RTG in combination with rivastigmine; FIG. 4B shows an overall decrease in the power of oscillations after 24 weeks of treatment with rivastigmine in combination with placebo. FIG. 5 reports the peak of significant increase in the range between 22 and 31 Hz for the combination of rotigotine and rivastigmine as compared to the decrease observed in the group of Ad patients treated with rivastigmine is combination with PCB.

In embodiments, the drug combination of rotigotine and rivastigmine administered to an AD patient includes a therapeutically effective dose of rotigotine ranging from about 2 to about 8 mg per 24 hours, and a complementary therapeutically effective dose of rivastigmine ranging from about 5 to about 23 mg per 24 hours.

While in the foregoing specification aspects of the present technologies have been described in relation to certain embodiments thereof, and many details have been put forth for the purpose of illustration, it will be apparent to those skilled in the art that the concepts and principles is susceptible to additional embodiments and that certain of the details described herein can be varied considerably without departing from the basic principles of the disclosure.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method for relieving symptoms of dementia associated with neurodegenerative diseases comprising: administering a transdermal combination drug formulation comprising a first therapeutically effective dose of rotigotine and a second complementary dose of an acetylcholinesterase inhibitor.

2. The method of claim 1, wherein the neurodegenerative disease comprises Alzheimer's disease, frontotemporal dementia or Lewy body disease, vascular dementia, or a combination of such findings.

3. The method of claim 2, wherein the acetylcholinesterase inhibitor comprises donepezil, galantamine huperzine or rivastigmine.

4. A method for relieving symptoms of dementia associated with neurodegenerative diseases comprising: administering a combination drug formulation comprising a first therapeutically effective dose of rotigotine and a complementary dose of a second drug.

5. The method of claim 4, wherein the combination drug formulation is administered orally, nasally, parenterally, topically, or transdermally.

6. The method of claim 5, wherein the neurodegenerative disease comprises Alzheimer's disease, frontotemporal dementia or Lewy body disease, vascular dementia, or a combination of such findings.

7. The method of claim 6, wherein the second drug comprises a cholinesterase inhibitor drug.

8. The method of claim 7, wherein the second drug comprises memantine.

9. A method for relieving agitation in a patient with a neurodegerative disease comprising: administering to the patient a transdermal combination drug formulation that delivers:
rotigotine in a dose from 2 mg/24 hours to 8 mg/24 hours; and
an acetylcholinesterase inhibitor in a dose from 5 mg to 23 mg per 24 hours.

10. The method of claim 9, wherein the neurodegenerative disease comprises Alzheimer's disease, frontotemporal dementia or Lewy body disease, vascular dementia, or a combination of such findings.

11. The method of claim 10, wherein the acetylcholinesterase inhibitor comprises donepezil, galantamine, huperzine or rivastigmine.
